(19) United States
(12) Patent Application Publication
(10) Pub. No.: US 2026/0025083 A1
(43) Pub. Date: Jan. 22, 2026

(54) ELECTROMAGNETIC STIMULATION APPARATUS AND BOOSTER DEVICE THEREOF

(71) Applicant: ADDON TECH LTD., TAIPEI CITY (TW)

(72) Inventors: SHIH-TU YEN, TAIPEI CITY (TW); YEN-LUNG LIN, TAIPEI CITY (TW)

(21) Appl. No.: 18/773,602

(22) Filed: Jul. 16, 2024

(51) Int. Cl. H02M 7/04; A61N 2/00; A61N 2/02

(52) U.S. Cl. CPC H02M 7/04; A61N 2/02; A61N 2/006

(57) ABSTRACT

An electromagnetic stimulation apparatus with a booster device using first and second current direction limiting elements and first and second energy storage elements to generate electromagnetic stimulation. The first current direction limiting element and the first energy storage element are electrically connected to an power supply in series, the first current direction limiting element is connected to the second current direction limiting element in parallel, and the second current direction limiting element is connected to the second energy storage element in series. The first and second current direction limiting elements are electrically connected or disconnected to switch the current direction and charge the first and second energy storage elements respectively at different periods. When charging the second energy storage element, the external power supply and the first energy storage element connected to the second energy storage element in series provide a cross-voltage doubling that of the first energy storage element.

### BACKGROUND OF THE DISCLOSURE

### Technical Field

The present disclosure relates to biological electromagnetic stimulation apparatus in general, and more particularly relates to a booster device and its electromagnetic stimulation apparatus that convert external AC current into DC high-voltage electricity with an intensity sufficient for an electromagnetic stimulation, in order to replace the related-art linear transformer while still maintaining electromagnetic stimulation performance and reducing equipment weight.

### Description of the Related Art

In order to study neuroscience and improve neuropsychiatric disorders, some electromagnetic stimulation apparatuses have been proposed. The electromagnetic stimulation apparatus uses alternating magnetic fields to stimulate electrical signals that affect various parts of a living organism, such as the transcranial magnetic stimulation (TMS) apparatus, which is a non-invasive neuromodulation technique that affects brain activity by generating varying magnetic fields through a magnetic field coil placed on the scalp of the living organism. In general, the critical stimulation parameters for each part of the organism will vary depending on the target site, the improvement solution, the depth of stimulation, and the neurological conditions. However, the electromagnetic stimulation apparatus commonly used in medical institutions still requires extremely high voltage to generate high current in order to effectively generate a stimulating magnetic field on the brain or nerves to activate the neurons and bring the nerves to action potentials. In the case of TMS, for example, a voltage of at least 300 volts (V) or even thousands of volts (V) is required to achieve the desired stimulation threshold, depending on the depth of the stimulation target and the condition of the nerves. In addition, since the targets of stimulation are the special characteristics of human body, the stimulation frequency range requires a better working frequency and fall in the 0.5-50 Hz in addition to the demand for high voltage and high current, so that it is difficult for the current common electromagnetic stimulation apparatus to use existing electronic transformers to satisfy the aforementioned requirements of high voltage, high current and working frequency, and the existing electronic transformers can only use the related-art linear transformers for the implementation. However, if this approach is chosen, the overall size and weight of the equipment cannot be miniaturized for general household use due to the design framework of the linear transformers. As a result, there is still a great deal of inconvenience for patients who need to seek frequent stimulation improvements and need to go to the medical institute repeatedly.

On the other hand, the related art has further proposed some other electromagnetic stimulation apparatuses, such as (1) the sacrificial intensity type is performed on the premise that the nerve cannot reach action potential, this type is generally not classified as a formal electromagnetic stimulation apparatus, and most of the current home electromagnetic stimulation apparatuses are of this type; (2) the sacrificial frequency type accumulates stimulation intensity in a weak charging speed and the frequency will not be more than 0.2 Hz. Although it can be applied to indications where extremely low frequencies are also useful, it is indeed a pioneer in successfully miniaturizing the electromagnetic stimulation apparatus. However, subject to the operating and output conditions, the adaptive symptoms of human body that can be stimulated and improved is limited; (3) the frequency overlap replaced intensity type with a booster device does not require a very high voltage to produce a biological effect by high-frequency continuous stimulation due to the overlap of biological effects although related researches suggested that low-intensity cannot directly cause biological effects and the booster devices of this type require a large amount of current for high-frequency output. Therefore, the choice other than the related-art linear transformer will not be adopted, and the issues of large volume and weight still exist. Today, even the simplest electromagnetic stimulation apparatus still has a total weight of approximately 20 kilograms and thus corresponding safety devices and rollers must be designed to facilitate its placement or transportation. If we take a 2000-watt conventional linear transformer as an example, the total weight of the transformer alone is about 15 kilograms, which is mainly due to the fact that the conventional linear transformers are basically composed of metal parts. In other words, 75% of the weight of the entire electromagnetic stimulation apparatus is due to the unavoidable presence of the large metal parts, which indirectly leads to one of the main reasons why the electromagnetic stimulation apparatus is still difficult to become popular or even to be used at home.

In view of the aforementioned drawbacks of the related art, the team of the present disclosure provides a light-weight electromagnetic stimulation apparatus with a booster device that is different from the learned mode of operation and capable of boosting the utility voltage up to 1 kV, and then making the device to output sufficient energy to drive the nerves to action potential and maintain the frequency above 0.5 Hz for continuous operation, in order to replace the related-art linear transformer and overcome the aforementioned drawbacks of the related art.

### SUMMARY OF THE DISCLOSURE

It is a primary objective of the present disclosure to overcome the problems of the related art by providing an electromagnetic stimulation apparatus with a booster device which uses the electronic properties of a basic voltage multiplier formed by two current direction limiting elements and two energy storage elements, and achieves a multi-voltage rectification and boosting effect through the structure with a series or parallel connection design to provide sufficient high-voltage DC for generating the required electromagnetic stimulation while maintaining the total weight for the lightweight effect.

To achieve the aforementioned objective, the present disclosure discloses a multi-stage booster device of an electromagnetic stimulation apparatus, which is electrically connected to an external power supply with a polar periodic change for a boost output application, characterized in that the multi-stage booster device is formed by a plurality of basic voltage multipliers coupled to one another, wherein the basic voltage multipliers comprise a first current direction limiting element, a first energy storage element, a second current direction limiting element and a second energy storage element; the external power supply is electrically coupled to the basic voltage multipliers, the first current direction limiting element, the first energy storage element of each basic voltage multiplier are electrically coupled to the external power supply in series, the first current direction limiting element is coupled in parallel to the second current direction limiting element with a current limiting direction opposite to that of the first current limiting direction, the second current direction limiting element is coupled to a second energy storage element in series, and the second energy storage element is situated between the first current direction limiting element and the second current direction limiting element; the first current direction limiting element and the second current direction limiting element of each basic voltage multiplier are connected or disconnected with the polarity of an input voltage for switching the current direction to drive the external power supply to charge the first energy storage element and the second energy storage element respectively at different periods, and during the process of charging the second energy storage element, the external power supply and the first energy storage element provide charging to make the cross-voltage of the second energy storage element to be double of that of the first energy storage element, and the second energy storage elements of the basic voltage multipliers are coupled to one another in series for an output application.

Wherein, the first energy storage element and the second energy storage element are capacitors, inductors, batteries, or any combination of the above. The second energy storage elements have a DC cross voltage exceeding 700 volts (V) after being connected to each other in series.

A secondary objective of the present disclosure is to disclose an electromagnetic stimulation apparatus including the aforementioned multi-stage booster device; and a stimulation unit electrically connected to the multi-stage booster device to achieve the effect of stimulating human body movements after boosting the voltage.

Wherein, the stimulation unit, as an output terminal of magnetic field, has an inductance value of at least 1 microhenry (μH). The stimulation unit is made of a copper wire coil or aluminum wire coil. The stimulation unit has a surface capable of generating a magnetic field exceeding 0.5 tesla (T) for at least one time. The stimulation unit has a surface capable of generating a magnetic field exceeding 0.1 tesla (T) and lasting less than 1 millisecond (ms) for more than three times consecutively.

In summation, the present disclosure uses the electronic properties and structural arrangement of components such as diodes or switches and capacitors or inductors to convert and boost a low voltage inputted from the external power supply into a high-voltage DC that is sufficient for the effective action potential of the stimulation unit. In other words, the present disclosure uses the arrangement and design of components to connect or disconnect each of the first and second current direction limiting elements according to the polarity of input voltage, so as to switch the energy storage period time of each of the first and second energy storage elements and make the cross-voltage of each second energy storage element to be twice of the DC voltage of the inputted peak value when the second energy storage element reaches a steady charging state. Further, the serially connected second energy storage elements form an overlapped cross-voltage for an output application. In this way, the multi-stage booster device realizes the multiple voltage boost function, and it is used to replace the related-art linear transformer while ensuring that the stimulation unit outputs sufficient intensity for biological stimulation, and improving the overall size and weight of the equipment. In addition, the cross-voltage overlap structure of the plurality of second energy storage elements can reduce the voltage stress that each energy storage element needs to withstand, so as to lower the cost of circuit components and improve the stability of circuits. In this way, the present disclosure breaks through the technical limitations of the related art and only uses common components such as diodes or switches and capacitors or inductors as the current direction limiting elements and energy storage elements, and through a special integration relationship of electrical connection, the present disclosure can meet the requirements for medical grade electromagnetic stimulation devices. At the same time, due to the selection of these components, the electromagnetic stimulation apparatus of the present disclosure has a small size, light weight and fast charge and discharge speed, while maintaining a better operating frequency and a lightweight electromagnetic stimulation apparatus.

### BRIEF DESCRIPTION OF THE DRAWINGS

FIG. 1 is a schematic block diagram showing the structure of a multi-stage booster device connected to an external power supply in accordance with a preferred embodiment of the present disclosure;

FIG. 2 is a circuit diagram showing a multi-stage booster device connected to an external power supply in accordance with a preferred embodiment of the present disclosure;

FIGS. 3A, 3B, 3C and 3D are circuit diagrams showing the charging of a multi-stage booster device connected to an external power in different period time in accordance with a preferred embodiment of the present disclosure; and

FIG. 4 is a schematic block diagram showing the structure of an electromagnetic stimulation apparatus connected to an external power supply in accordance with another preferred embodiment of the present disclosure.

### DETAILED DESCRIPTION OF THE PREFERRED EMBODIMENTS

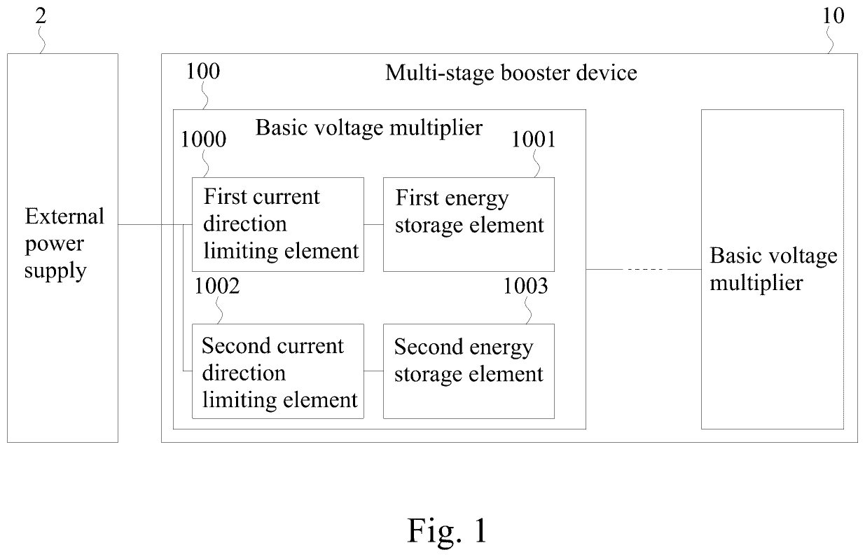

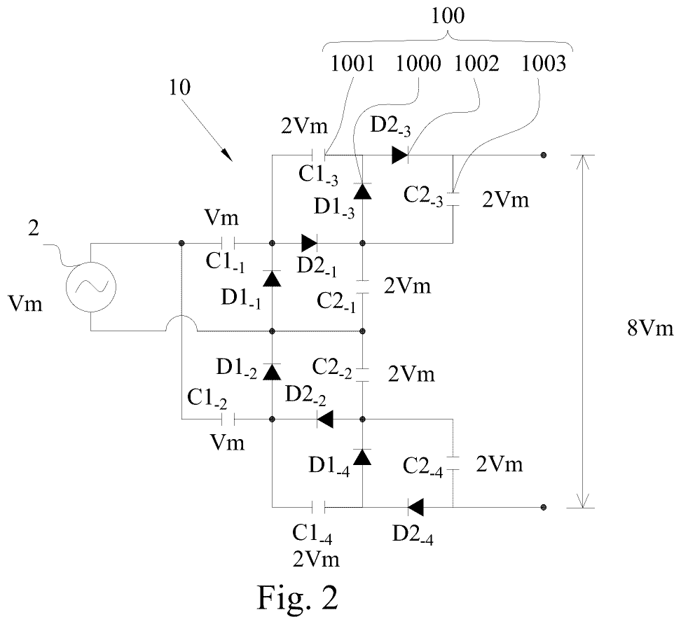

In order to enable those having ordinary skill in the art to clearly understand the contents of the present disclosure, the following descriptions and drawings are provided for reference. With reference to FIGS. 1 and 2 for the schematic block diagram and the circuit diagram showing a multi-stage booster device connected to an external power supply in accordance with a preferred embodiment of the present disclosure respectively, the multi-stage booster device 1 of the electromagnetic stimulation apparatus is electrically connected to an external power supply 2 with a polar periodic change for a boost output application, and the multi-stage booster device 1 is formed by a plurality of basic voltage multipliers 100. Further, each of the basic voltage multipliers 100 includes a first current direction limiting element 1000, a first energy storage element 1001, a second current direction limiting element 1002 and a second energy storage element 1003. The external power supply 2 is electrically connected to the basic voltage multipliers 100, and the first current direction limiting element 1000 and the first energy storage element 1001 of each basic voltage multiplier 100 are electrically connected to the external power supply 2 in series, the first current direction limiting element 1000 is connected in parallel to the second current direction limiting element 1002 having a current direction opposite to that of the first current direction limiting element, the second current direction limiting element 1002 is connected to a second energy storage element 1003 in series, so that the second energy storage element 1003 is situated between the first current direction limiting element 1000 and the second current direction limiting element 1002.

With the above electrical structure, the first current direction limiting element 1000 and the second current direction limiting element 1002 of each basic voltage multiplier 100 are connected or disconnected with the polarity of the input voltage for switching the current direction to drive the external power supply 2 to charge the first energy storage element 1001 and the second energy storage element 1003 respectively at different periods, and during the process of charging the second energy storage element 1003, the external power supply 2 and the first energy storage element 1001 provide charging to change the cross-voltage of the second energy storage element 1003 to twice of that of the first energy storage element 1001, and the second energy storage elements 1003 of the basic voltage multipliers 100 are connected to one another in series. In other words, the rules of the above electrical connection can increase the quantity of basic voltage multipliers 100 continuously, and the cross-voltage value suitable for the output application of terminals can be selected based on this after boosting.

In this embodiment, the first energy storage element 1001 and the second energy storage element 1003 are capacitors, inductors, batteries or any combination of the above, and the first current direction limiting element 1000 and the second current direction limiting element 1002 are diodes, switches or their combination, such that the second energy storage elements 1003 have a DC cross-voltage exceeding 700 volts (V) after being connected in series. For example, the basic voltage multipliers 100 as shown in FIG. 2 can use the structure of each first energy storage element 1001 and each second energy storage element 1003 which are capacitors and each first current direction limiting element 1000 and each second current direction limiting element 1002 which are diodes to form the multi-stage booster device 10 with 8 times of voltage, which includes the following four basic voltage multipliers 100:

### 1. The First Basic Voltage Multiplier 100:

Two terminals of the first one of the first energy storage elements 1001 (C1₋₁) are coupled to a terminal of the external power supply 2 and a terminal of the first one of the first current direction limiting elements 1000 (D1₋₁), and another terminal of the D1₋₁ is coupled to another terminal of the external power supply 2, so that the D1₋₁ and the C1₋₁ present an electrical connection in series with the external power supply 2; at the same time, a terminal of the first one of the second current direction limiting elements 1002 (D2₋₁) is coupled to a connection point between the C1₋₁ and the D1₋₁, another terminal of the D2₋₁ is coupled to a terminal of the first one of the second energy storage elements 1003 (C2₋₁) and the D2₋₁ is connected to the D1₋₁ in parallel and has an opposite current limiting direction, and another terminal of the C2₋₁ is coupled to a connection point between the D1₋₁ and the external power supply 2, so that the D2₋₁ is connected to the C2₋₁ in series and the C2₋₁ is situated between the D1₋₁ and the D2₋₁. In this way, the first one of the basic voltage multipliers 100 with a first voltage doubler circuit is assembled and formed. If the external power supply 2 is mains power that supplies a sine wave AC with a peak voltage value of Vm, two terminals of the C2₋₁ have a cross-voltage of 2Vm.

### 2. The Second Basic Voltage Multiplier 100 Connected to the First Basic Voltage Multiplier 100:

By the same circuit structure as above, a second voltage doubler circuit connected to the first one of the basic voltage multipliers 100 is assembled and formed. Two terminals of the second one of the first energy storage elements 1001 (C1₋₂) are coupled between the external power supply 2 and the C1₋₁ and a terminal of the second one of the first current direction limiting elements 1000 (D1₋₂), and another terminal of the D1₋₂ is coupled between the external power supply 2 and the D1₋₁; at the same time, a terminal of the second one of the second current direction limiting elements 1002 (D2₋₂) is coupled to a connection point between the C1₋₂ and the D1₋₂, another terminal of the D2₋₁ is coupled to a terminal of the second one of the second energy storage elements 1003 (C2₋₂), and another terminal of the C2₋₂ is coupled between the C2₋₁, the D1₋₁ and the D1₋₂, such that two terminals of the C2₋₂ have a cross-voltage of 2Vm.

### 3. The Third Basic Voltage Multiplier 100 Connected to the First Basic Voltage Multiplier 100:

Continuously, a third voltage boost circuit is assembled and formed. Two terminals of the third one of the first energy storage elements 1001 (C1₋₃) are coupled between the C1₋₁, the D1₋₁ and the D2₋₁ and a terminal of the third one of the first current direction limiting elements 1000 (D1₋₃), another terminal of the D1₋₃ is coupled between the D2₋₁ and the C2₋₁; at the same time, a terminal of the third one of the second current direction limiting elements 1002 (D2₋₃) is coupled to a connection point between the C1₋₃ and the D1₋₃, another terminal of the D2₋₃ is coupled to a terminal of the third one of the second energy storage elements 1003 (C2₋₃), and another terminal of the C2₋₃ is coupled between the C2₋₁, the D2₋₁ and the D1₋₃, such that two terminals of the C2₋₃ have a cross-voltage of 2Vm.

### 4. The Fourth Basic Voltage Multiplier 100 Connected to the Second Basic Voltage Multiplier 100:

A fourth voltage doubler circuit is assembled and formed. Two terminals of the fourth one of the first energy storage elements 1001 (C1₋₄) are coupled between the C1₋₂, the D1₋₂ and the D2₋₂ and a terminal of the fourth one of the first current direction limiting elements 1000 (D1₋₄), and another terminal of the D1₋₄ is coupled between the D2₋₂ and the C2₋₂; at the same time, a terminal of the fourth one of the second current direction limiting elements 1002 (D2₋₄) is coupled to a connection point between the C1₋₄ and the D1₋₄, another terminal of the D2₋₄ is coupled to a terminal of the fourth one of the second energy storage elements 1003 (C2₋₄), and another terminal of the C2₋₄ is coupled between the C2₋₂, the D2₋₂ and the D1₋₄, such that two terminals of the C2₋₄ have a cross-voltage of 2Vm.

In this way, the voltage value outputted by the multi-stage booster device 10 is a cross-voltage formed by connecting the second energy storage elements 1003 of the C2₋₃, C2₋₁, C2₋₂ and C2₋₄ in series, and thus the multi-stage booster device 10 can output a voltage value of 8Vm without considering losses. If the external power supply supplies a mains power of 110 VAC, and the peak voltage Vm reaches 155 VDC, a direct current of approximately 1200V can be output after the operation of the multi-stage booster device 10, thereby increasing the small voltage inputted by the external power supply 2 to a high voltage that meets the requirements of the electromagnetic stimulation apparatus 1.

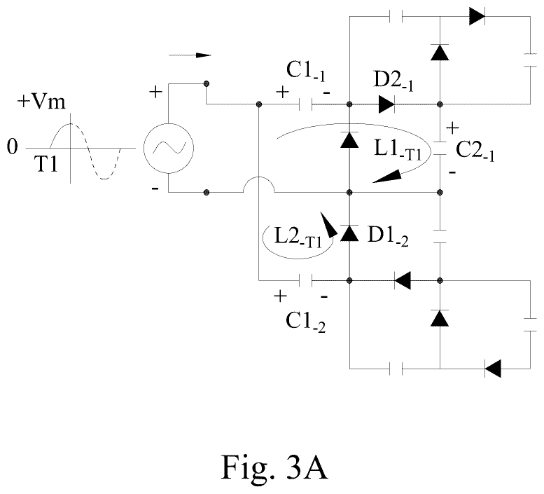

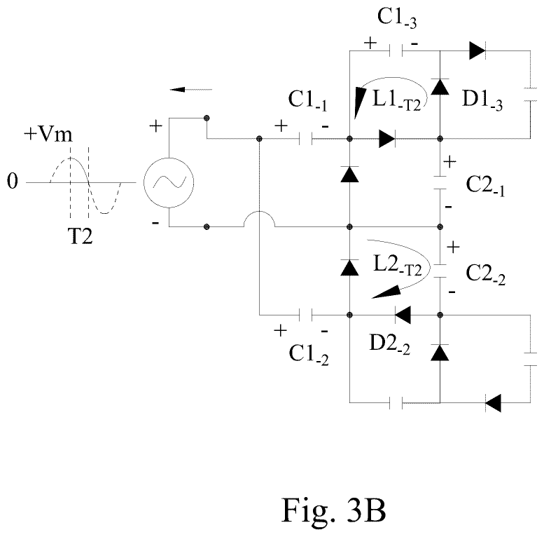

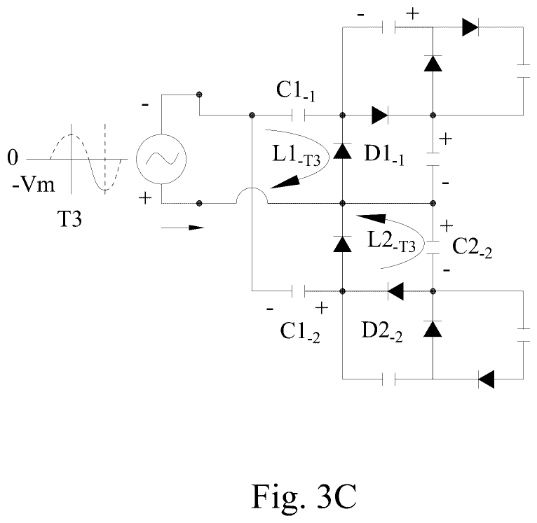

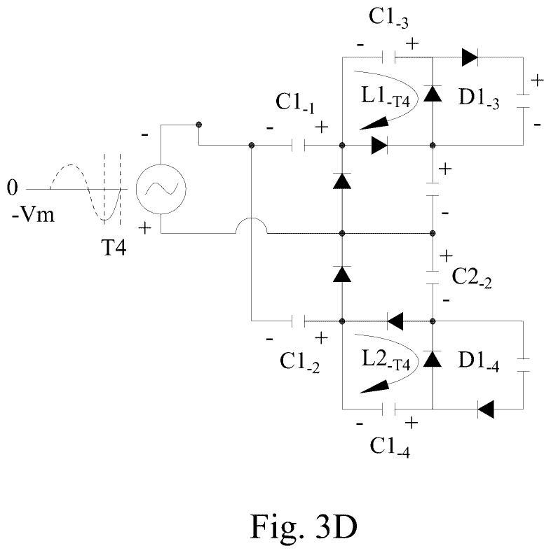

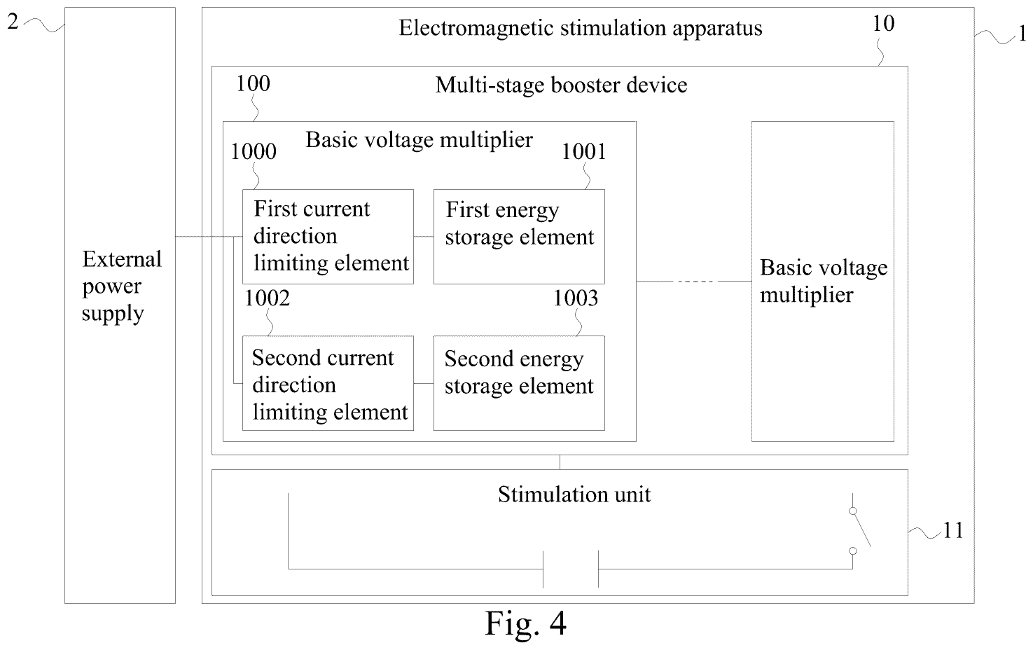

With reference to FIGS. 3A to 3D for the circuit diagrams showing the charging of a multi-stage booster device connected to an external power in different period time in accordance with a preferred embodiment of the present disclosure, the AC voltage supplied by the external power supply 2 is based on a complete sine wave as a period (T) with a sine wave period of T1+T2 and a negative sine wave period of T3+T4, so that the charging process of the basic voltage multipliers 100 in each period time is as follows:

### 1. In the Period Time T1 as Shown in FIG. 3A:

The AC voltage supplied by the external power supply 2 rises from 0 to the peak +Vm, such that when the input terminal of the multi-stage booster device 10 gradually generates a voltage difference, the D2₋₁ and the D1₋₂ are conducted, so that the external power supply 2, C1₋₁, D2₋₁ and C2₋₁ constitute a positive cycle charging loop L1_{-T1}, while the external power supply 2, C1₋₂ and D1₋₂ constitute a positive cycle charging loop L2_{-T1}. Accordingly, current flows into the C1₋₁, C2₋₁ and C1₋₂ until the end of the period time T1, causing the C1₋₁ and C2₋₁ to store energy to half peak value and the C1₋₂ to store energy to peak value.

### 2. In the Period Time T2 as Shown in FIG. 3G:

The AC voltage supplied by the external power supply 2 drops from peak +Vm to 0, and at this time, the D1₋₃ and the D2₋₂ are conducted due to the characteristics of the components, and current flows from C2₋₁ into the C1₋₃, and flows from the C1₋₂ into C2₋₂ until the end of the period time T2, and the C1₋₁, C2₋₁, C1₋₃, C1₋₂ and C2₋₂ can reach potential energy balance. In other words, in the period time T2, when the voltage difference between two input terminals of the multi-stage booster device 10 coupled to the external power supply 2 decreases, the external power supply 2, C1₋₁, C2₋₁, D1₋₃ and C1₋₃ constitute a positive cycle balancing loop L1_{-T2}, and at the same time, the external power supply 2, C1₋₂, C2₋₂ and D2₋₂ constitute a positive cycle balance loop L2_{-T2}.

### 3. In the Period Time T3 as Shown in FIG. 3C:

The AC voltage supplied by the external power supply 2 drops from 0 to the valley-Vm, such that when two input terminals of the multi-stage booster device 10 generate a voltage difference again, the D1₋₁ and the D2₋₂ are conducted due to the characteristics of the components, so that the external power supply 2, C1₋₁ and D1₋₁ constitute a negative cycle charging loop L1_{-T3}, and at the same time, the external power supply 2, C1₋₂, D2₋₂ and C2₋₂ constitute a negative cycle charging loop, so that current flows into the C1₋₁, C1₋₂ and C2₋₂, and the C1₋₁ stores energy to the peak value and the C1₋₂ and C2₋₂ store energy to the half peak value.

### 4. In the Period Time T4 as Shown in FIG. 3D:

The AC voltage supplied by the external power supply 2 rises from the valley −Vm to 0, the D1₋₃ and the D14 are conduced due to the characteristics of the components, so that current flows from the C1₋₁ into the C1₋₃, and flows from the C2₋₂ into the C1₋₄ until the end of the period time T4, and the C1₋₁, C1₋₃, C1₋₂, C2₋₂ and C1₋₄ reach potential energy balance. In other words, in the period time T4, the voltage difference at the input terminal of the multi-stage booster device 10 decreases, the external power supply 2, C1₋₁, C1₋₃ and D1₋₃ constitute a negative cycle balance loop L1_{-T4}, and at the same time, the external power supply 2, C1₋₂, C2₋₂, D1₋₄ and C1₋₄ constitute a negative cycle balance loop L2_{-T4}.

From this, we know that as the period time T1˜T4 progresses, each first and second energy storage elements 1001, 1003 in each basic voltage multiplier 100 will sequentially store energy to a stable state. In this way, the external power supply 2 is used to input the AC sine wave for the period time T1˜T4 until it reaches the stable state, and the capacitance and voltage of each first energy storage element 1001 and each second energy storage element 1003 will reach the equilibrium of potential energy, and the C2₋₃, C2₋₁, C2₋₂ and C2₋₄ are changed to twice of the peak input voltage Vm, and the value of voltage outputted by the multi-stage booster device 10 is the cross-voltage of the serially connected second energy storage elements 1003 which is the DC voltage of 8Vm. It is noteworthy that even if a higher output voltage value is required due to actual conditions, by using the basic voltage multipliers 100 of the present disclosure, it is still easy to further connect and extend one, two or more basic voltage multipliers 100 in the circuit of the existing multi-stage booster device 10, thereby expanding the voltage output capability of the multi-stage booster device 10. Furthermore, by using this series circuit structure to achieve an eight-times voltage boost, the voltage stress that each energy storage element needs to withstand can be greatly reduced, thereby reducing the overall circuit architecture cost and improving circuit stability. Further, another objective of the present disclosure is to use the electromagnetic stimulation apparatus 1 assembled with the multi-stage booster device 10 at home or similar places. In FIG. 5, the electromagnetic stimulation apparatus 1 includes the multi-stage booster device 10 and a stimulation unit 11, where the stimulation unit 11 is a copper wire coil or aluminum wire coil electrically connected to the multi-stage booster device 10 for capturing the DC voltage outputted by the multi-stage booster device 10 to generate the actuating electric power. It is noteworthy that the inductance of the stimulation unit 11 serving as the output terminal of magnetic field is set to at least one microhenry (μH) in conjunction with the cross-voltage value outputted by the multi-stage booster device 10 in order to provide a better performance of the electromagnetic stimulation apparatus 1, and the stimulation unit 11 generates a magnetic field of more than 0.5 tesla (T) for at least one time on its surface, or the stimulation unit 11 continuously generates a magnetic field of more than 0.1 tesla (T) and lasting less than 1 millisecond (ms) for at least three times on its surface, thereby improving the adaptability of the human body. Specifically, the stimulation unit 11 can be released after being fully charged through the energy storage device connected to the front end, thereby achieving a very large current change, and it can be used to generate the magnetic field with the corresponding specifications for stimulation as described above.

In summation of the description above, the present disclosure uses the electronic properties and structural arrangement of components such as diodes or switches and capacitors or inductors to convert and boost a low voltage inputted from the external power supply into a high-voltage DC that is sufficient for the effective action potential of the stimulation unit. In other words, the present disclosure uses the arrangement and design of components to connect or disconnect each of the first and second current direction limiting elements according to the polarity of input voltage, so as to switch the energy storage period time of each of the first and second energy storage elements and make the cross-voltage of each second energy storage element to be twice of the DC voltage of the inputted peak value when the second energy storage element reaches a steady charging state. Further, the serially connected second energy storage elements form an overlapped cross-voltage for an output application. In this way, the multi-stage booster device realizes the multiple voltage boost function, and it is used to replace the related-art linear transformer while ensuring that the stimulation unit outputs sufficient intensity for biological stimulation, and improving the overall size and weight of the equipment. In addition, the cross-voltage overlap structure of the plurality of second energy storage elements can reduce the voltage stress that each energy storage element needs to withstand, so as to lower the cost of circuit components and improve the stability of circuits. In this way, the present disclosure breaks through the technical limitations of the related art and only uses common components such as diodes or switches and capacitors or inductors as the current direction limiting elements and energy storage elements, and through a special integration relationship of electrical connection, the present disclosure can meet the requirements for medical grade electromagnetic stimulation devices. At the same time, due to the selection of these components, the electromagnetic stimulation apparatus of the present disclosure has a small size, light weight and fast charge and discharge speed, while maintaining a better operating frequency and a lightweight electromagnetic stimulation apparatus.

What is claimed is:

1. A multi-stage booster device of an electromagnetic stimulation apparatus, electrically coupled to an external power supply with a polar periodic change for a boost output application, characterized in that:
the multi-stage booster device is formed by a plurality of basic voltage multipliers coupled to one another, wherein the plurality of basic voltage multipliers comprises a first current direction limiting element, a first energy storage element, a second current direction limiting element, and a second energy storage element;
the external power supply is electrically coupled to the plurality of basic voltage multipliers, the first current direction limiting element and the first energy storage element of each of the plurality of basic voltage multipliers are electrically coupled to the external power supply in series, the first current direction limiting element is coupled in parallel to the second current direction limiting element with a current limiting direction opposite to a current limiting direction of the first current limiting direction limiting element, the second current direction limiting element is coupled to the second energy storage element in series such that the second energy storage element is situated between the first current direction limiting element and the second current direction limiting element;
the first current direction limiting element and the second current direction limiting element of each of the plurality of basic voltage multipliers are connected or disconnected with a polarity of an input voltage for switching a current direction to drive the external power supply to charge the first energy storage element and the second energy storage element, respectively, at different periods, and during a process of charging the second energy storage element, the external power supply and the first energy storage element provide charging to make a cross-voltage of the second energy storage element to be double of that of the first energy storage element, and the second energy storage elements of the plurality of basic voltage multipliers are coupled to one another in series for an output application.

2. The multi-stage booster device according to claim 1, wherein the first energy storage element and the second energy storage element are capacitors, inductors, batteries, or a combination thereof.

3. The multi-stage booster device according to claim 1, wherein the first current direction limiting element and the second current direction limiting element are diodes, switches, or a combination thereof.

4. The multi-stage booster device according to claim 1, wherein the second energy storage elements have a DC cross-voltage exceeding 700 volts (V) after being coupled to one another in series.

5. An electromagnetic stimulation apparatus, comprising:
the multi-stage booster device according to claim 1; and
a stimulation unit electrically coupled to the multi-stage booster device.

6. The electromagnetic stimulation apparatus according to claim 5, wherein the stimulation unit, as an output terminal of a magnetic field, has an inductance value of at least 1 microhenry (μH).

7. The electromagnetic stimulation apparatus according to claim 5, wherein the stimulation unit is made of a copper wire coil or aluminum wire coil.

8. The electromagnetic stimulation apparatus according to claim 5, wherein the stimulation unit has a surface capable of generating a magnetic field exceeding 0.5 tesla (T) for at least one time.

9. The electromagnetic stimulation apparatus according to claim 5, wherein the stimulation unit has a surface capable of generating a magnetic field exceeding 0.1 tesla (T) and lasting less than 1 millisecond (ms) for more than three times consecutively.
